(19) United States
(12) Patent Application Publication
(10) Pub. No.: US 2014/0220097 A1
(43) Pub. Date: Aug. 7, 2014

(54) Method Of Implanting A Sterile, Active Agent-Coated Material And Composition Made According To Same

(71) Applicant: DePuy Synthes Products, LLC, Raynham, MA (US)

(72) Inventor: Maria Maccecchini, Berwyn, PA (US)

(73) Assignee: DePuy Synthes Products, LLC, Raynham, MA (US)

(21) Appl. No.: 14/256,054

(22) Filed: Apr. 18, 2014

Related U.S. Application Data
(63) Continuation of application No. 11/108,040, filed on Apr. 15, 2005.
(60) Provisional application No. 60/563,014, filed on Apr. 19, 2004.

(51) Int. Cl. A61K 9/00; A61K 38/18

(52) U.S. Cl. CPC A61K 9/0024; A61K 38/18; A61K 38/1858; A61K 38/1841
USPC 424/423; 514/8.5; 514/8.2; 514/8.9; 424/422

(57) ABSTRACT

The present invention relates to a method of implanting a sterile, active agent-coated material comprising contacting a sterile implant with a sterile active agent or active agent solution to form a sterile, active agent-coated implant and, at most a relatively short time after forming the active agent-loaded sterile implant, implanting the active agent-loaded sterile implant into a subject such as a mammal or a human.

### CROSS-REFERENCE TO RELATED APPLICATIONS

The present invention claims priority to Provisional Application No. 60/563,014, filed Apr. 19, 2004, which is incorporated in its entirety by reference.

### FIELD OF THE INVENTION

The present invention relates to a method of implanting a sterile, active agent-coated material comprising contacting a sterile implant with a sterile active agent or active agent solution to form a sterile, active agent-coated implant and, at most a relatively short time after forming the active agent-loaded sterile implant, implanting the active agent-loaded sterile implant into a subject.

### BACKGROUND OF THE INVENTION

Bony defects, whether from degenerative, traumatic or cancerous etiologies, pose a formidable challenge to the reconstructive surgeon. Particularly difficult is reconstruction or repair of skeletal parts that comprise part of a multi-tissue complex, such as occurs in mammalian joints.

Mammalian bone tissue is known to contain one or more proteinaceous materials presumably active during growth and natural bone healing which can induce a developmental cascade of cellular events resulting in endochondral bone formation. The developmental cascade involved in endochondral bone differentiation consists of chemotaxis of mesenchymal cells, proliferation of progenitor cells into chondrocytes and osteoblasts, differentiation of cartilage, vascular invasion, bone formation, remodeling, and finally marrow differentiation.

True osteogenic factors capable of inducing the above-described cascade of events that result in endochondral bone formation have now been identified, isolated, and cloned. These morphogenic and mitogenic proteins, which can also occur in nature as peptides, are referred to in the art as “osteogenic” proteins, “osteoinductive” proteins, and “bone morphogenetic” proteins. Whether naturally-occurring or synthetically prepared, these osteogenic proteins, when implanted in a mammal typically in association with a substrate that allows the attachment, proliferation and differentiation of migratory progenitor cells, are capable of inducing recruitment of accessible

progenitor cells and stimulating their proliferation, inducing differentiation into chondrocytes and osteoblasts, and further inducing differentiation of intermediate cartilage, vascularization, bone formation, remodeling, and finally marrow differentiation. Those proteins are referred to as members of the Vgr-1/OP1 protein subfamily of the TGF-β super gene family of structurally related proteins. Members include the proteins described in the art as OP1 (BMP-7), OP2 (BMP-8), BMP2, BMP3, BMP4, BMP5, BMP6, 60A, DPP, Vgr-1 and Vgl. See, e.g., U.S. Pat. Nos. 5,011,691 and 5,266,683; Ozkaynak et al. (1990) EMBO J. 9:2085-2093; Wharton et al. (1991) PNAS, 88:9214-9218; Ozkaynak (1992) J. Biol. Chem., 267:25220-25227; Celeste et al. (1991) PNAS, 87:9843-9847; and Lyons et al. (1989) PNAS, 86:4554-4558. These disclosures describe the amino acid and DNA sequences, as well as the chemical and physical characteristics of these proteins. See also Wozney et al. (1988) Science, 242:1528-1534; International Publication No. WO93/00432 (BMP 9); Padgett et al. (1987) Nature, 325:81-84 (DPP); and Weeks (1987) Cell, 51:861-867 (Vg-1).

However, to date, obtaining solid implants that are loaded with active agents such as osteogenic proteins have been difficult, in part due to sterility and shelf-life issues. Disadvantages associated with prior art active agent-loaded implantable devices include, the limited shelf life of such devices, the fact the active agent is easily degraded when the device is sterilized (e.g., by heat or by exposure to ethylene dioxide), and the inability of a physician to alter the dosage to which a patient is subjected by implantation of the device. Additionally, the cost of prior art active agent-loaded implantable devices has been very high, as it necessarily includes the costs associated with the stringent regulatory requirements attendant a drug containing device.

The prior art has been unable to overcome these disadvantages and shortcomings and a new approach is needed to safely, effectively, and economically deliver an active agent to an implantation site. The present invention, as described below, offers one such approach.

### SUMMARY OF THE INVENTION

One aspect of the invention relates to a method of forming a sterile active agent-loaded implant including: providing a sterile implant, optionally having a coating layer, having a surface that is capable of physically and/or chemically associating with an active agent; providing a sterile composition containing an active agent and optionally a carrier, a solvent, or both; and contacting the sterile implant or coating layer surface with the sterile active agent-containing composition, so that the sterile implant at least partially physically and/or chemically associates at least with the active agent, and optionally with the carrier and/or solvent, thus forming an active agent-loaded sterile implant.

Advantageously, the sterile active agent-containing composition can include a carrier, a solvent, or both. In one embodiment, the solvent comprises water. In another embodiment, the active agent can include an antibacterial agent; an antiviral agent; an osseointegrative, osteoconductive, and/or osteoinductive agent; or a combination thereof. In another embodiment, the active agent can include a mitogenic growth factor, a morphogenic growth factor, an osteoclast inhibitor, an antiinflammatory agent, or a combination thereof.

In still another embodiment, the coating layer comprises hyaluronic acid, a hyaluronate salt, a (co)polymer containing alkylene oxide repeat units, or a combination thereof. In yet another embodiment, the coating layer comprises a hydrophilic (co)polymer, a water-swellable (co)polymer, or both.

In another embodiment, the step of providing the sterile implant includes sterilization of a non-sterile implant through exposure to heat, radiation, chemical treatment, or a combination thereof. In another embodiment, the step of providing the sterile active agent-containing composition includes sterilization of the non-sterile active agent-containing composition through exposure to heat, filtration through a sub-micron filter, or a combination thereof.

Another aspect of the invention relates to a method of implanting a sterile active agent-coated material and/or device including: performing the method described above to form an active agent-loaded sterile implant; and at most a relatively short time after forming the active agent-loaded sterile implant, implanting the active agent-loaded sterile implant into a subject.

Advantageously, the time between forming the active agent-loaded sterile implant and implanting the active agent-loaded sterile implant into a subject can be from about 45 seconds to about 3 hours.

In one embodiment, the sterile active agent-containing composition can include a carrier, a solvent, or both. In another embodiment, the solvent comprises water. In another embodiment, the active agent can include an antibacterial agent; an antiviral agent; an osseointegrative, osteoconductive, and/or osteoinductive agent; or a combination thereof. In another embodiment, the active agent can include a mitogenic growth factor, a morphogenic growth factor, an osteoclast inhibitor, an antiinflammatory agent, or a combination thereof.

In still another embodiment, the coating layer comprises hyaluronic acid, a hyaluronate salt, a (co)polymer containing alkylene oxide repeat units, or a combination thereof. In yet another embodiment, the coating layer comprises a hydrophilic (co)polymer, a water-swellable (co)polymer, or both.

In another embodiment, the step of providing the sterile implant includes sterilization of a non-sterile implant through exposure to heat, radiation, chemical treatment, or a combination thereof. In another embodiment, the step of providing the sterile active agent-containing composition includes sterilization of the non-sterile active agent-containing composition through exposure to heat, filtration through a sub-micron filter, or a combination thereof.

These and other features and advantages of the present invention will become apparent from the remainder of the disclosure, in particular the following detailed description of the preferred embodiments, all of which illustrate by way of example the principles of the invention.

### DETAILED DESCRIPTION OF THE PREFERRED EMBODIMENTS

One aspect of the present invention relates to a method of implanting a sterile, active agent coated material and/or device for implantation into a subject. Advantageously, the method can comprise, but is not limited to, the following steps: providing a sterile implantable material and/or device (hereinafter “sterile implant,” for convenience only and without intent to limit) that is capable of physically and/or chemically associating with an active agent; contacting the sterile implant with an active agent, e.g., by at least partially exposing the sterile implant to a preferably sterile solution containing the active agent, so that the sterile implant at least partially physically and/or chemically associates with the active agent, thus forming an active agent-loaded sterile implant; and at most a relatively short time after forming the active agent-loaded sterile implant, implanting the active agent-loaded sterile implant into a subject, e.g., an animal such as a mammal, preferably a primate or a human.

Before a sterile implant can be provided, typically a non-sterile implant is formed. This non-sterile implant may comprise, or be made from, any suitable material, preferably a biocompatible material, and optionally but preferably a bioabsorbale, bioresorbable, and/or biodegradable material. Exemplary implant materials can include, but are not limited to: natural and/or synthetic (co)polymers; metals; metal alloys; glasses (e.g., bioactive glasses such as E-glass); metal-containing compounds such as metal oxides (e.g., ceramics), hydroxides, carbonates, nitrates, phosphates, sulfates, and the like; and the like; and combinations thereof.

The natural and/or synthetic polymers can be thermoplastic or thermoset, elastic or viscoelastic, elastomeric or non-elastomeric, semi-crystalline or amorphous, oriented or unoriented, hydrogen-bonded, or non-hydrogen-bonded, and the like, depending upon the application for which they are to be used. Natural and/or synthetic polymers can be homopolymers, blends of homopolymers, copolymers, blends of copolymers, or blends of homopolymers and copolymers. If homopolymeric, the natural and/or synthetic polymers can be, but are not limited to being, atactic, isotactic, syndiotactic, dendritic, long-chain branched/grafted, short-chain branched/hairy-rodlike, uncrosslinked, crosslinked, multi-armed stars, or the like, or some combination thereof. If copolymeric, the natural and/or synthetic polymers can include, but are not limited to, block copolymers (e.g., diblock or triblock), multiblock copolymers, long- and/or short-chain graft copolymers, long- and/or short-chain multigraft copolymers, long- and/or short-chain comb copolymers, random copolymers, alternating copolymers, hetero-armed star copolymers, diblock armed star copolymers, triblock armed star copolymers, multiblock armed star copolymers, and the like, and combinations or copolymers thereof. Copolymers according to the invention may contain two different types of repeat units or may contain more than two different types of repeat units (e.g., terpolymers contain three different types). (Co)Polymers according to the invention are preferably designated according to the process of their synthesis and not necessarily according to the end product (e.g., a completely hydrogenated polyisoprene is preferably characterized as a hydrogenated polyisoprene homopolymer and preferably not as an alternating ethylene-propylene copolymer).

Examples of natural and synthetic polymers include, but are not limited to, (co)polymers containing repeat units and/or (co)polymers made including precursors (i.e., monomers, dimers, oligomers, and the like, and combinations thereof) of aliphatic ethers (such as methylene oxide, ethylene oxide, propylene oxide, tetramethylene oxide, and the like, and copolymers and combinations thereof), aliphatic esters (such as caprolactones, e.g., ε-caprolactone; alkylene esters, e.g., ethylene adipate, butylene adipate, ethylene succinate, ethylene sebacate, ethylene glutarate, lactides/lactic acids (such as D-, L-, D,L-, and the like, and copolymers and combinations thereof), glycolides/glycolic acids, and the like, and combinations or copolymers thereof; and the like; and copolymers and combinations thereof), aromatic esters (such as ethylene terephthalate, butylene terephthalate, isophthalates, and the like, and copolymers and combinations thereof), aliphatic amides (such as lactams, e.g., propiolactam, caprolactam, laurolactam, and the like, and combinations and copolymers thereof; polyamides, e.g., nylon 6,6, nylon 6,9, nylon, 6,10, nylon 6,12, and the like; and copolymers and combinations thereof), siloxanes (such as alkyl and/or dialkyl siloxanes, e.g., methylsiloxane, dimethylsiloxane, methylethylsiloxane, and the like, and combinations and copolymers thereof), urethanes and/or urethaneureas having hard segments made from at least diisocyanates (such as methylene diphenylene diisocyanate (MDI), methylene bis(cyclohexane isocyanate) (H₁₂MDI), isophorone diisocyanate (IPDI), phenylene diisocyanate, cyclohexane diisocyanate, toluene diisocyanate (TDI), methylcyclohexane diisocyanate, or the like, or combinations thereof) in combination with either diols (such as ethylene glycol, propylene glycol, butylene glycol, hexamethylene glycol, dihydroxybenzene, or the like, or a combination thereof) or diamines (such as ethylenediamine, propylenediamine, hexamethylenediamine, diaminocyclohexane, aniline, or the like, or a combination thereof) or both, optionally also including trifunctional and/or tetrafunctional components (such as triisocyanates, tetraisocyanates, triols, tetrols, triamines, tetramines, or the like, or a combination thereof) to chemically crosslink the (co)polymer system, alpha-olefins such as polyethylene (particularly UHMWPE), at least partially halogenated (particularly fluorinated) repeat units (e.g., vinyl halide, vinylidene halide, tetrahaloethylene, hexahalopropylene, perhaloalkoxy monomers such as those that form the commercial (co)polymer PFA available from DuPont of Wilmington, Del., perhaloester monomers, and the like, and combinations and copolymers thereof), ionomers (such as those that form the commercial (co)polymer SURLYN available from DuPont of Wilmington, Del., and the like), and the like, and combinations or copolymers thereof.

Metals and metal alloys useful as implant surfaces in the present invention are preferably non-toxic, preferably biocompatible, and can include, but are not limited to, titanium, chromium, manganese, cobalt, nickel, zinc, molybdenum, ruthenium, silver, tin, tantalum, gold, and the like, and combinations and alloys thereof, optionally with non-enumerated metals. In one embodiment, the metal or metal alloy can contain titanium, silver, and/or gold.

Metal-containing compounds useful as implant surfaces in the present invention are also preferably non-toxic, preferably biocompatible, and are preferably metal-containing carbides, carbonates, nitrides, nitrites, nitrates, oxides, oxynitrides, hydroxides, phosphides, phoshites, phosphates, sulfides, sulfites, sulfates, or combinations thereof. The reacted metals can include, but are not limited to, the following metals: beryllium, boron, magnesium, aluminum, calcium, titanium, vanadium, chromium, manganese, iron, cobalt, nickel, copper, zinc, strontium, zirconium, molybdenum, ruthenium, tin, barium, tantalum, and the like, and combinations thereof, optionally with non-enumerated metals. In one embodiment, the reacted metal can include one or more metals of Group IIA of the periodic table. In another embodiment, the reacted metal can include one or more of the aforementioned transition metals.

The kind of implant used in the process according to the invention is not limited and can take any appropriate form and shape desired for and/or required by the application for which it will ultimately be used. Examples of useful implants, therefore, include, but are not limited to: screws (e.g., bone screws, pedicle screws, or the like); tacks; nails (e.g., intramedullary nails, soft-tissue anchoring nails, or the like); pins (e.g., bone pins, immobilizer pins, or the like); plates (e.g., bone plates, maxillofacial plates, or the like); rods; clamps; staples; springs; stents; sutures; membranes (e.g., for protecting bones or portions thereof; for protecting osteogenic implant compositions, or the like); catheters; pacemaker or other electronic device leads; xenograft, heterograft, or allograft portions of bone, soft tissue (e.g., muscle), extracellular matrix (ECM, e.g., collagen), cartilagenous material (e.g., joints such as knee, intervertebral discs, ears, noses, or the like), or the like, or combinations thereof; compositions of or containing artificial bone, soft tissue (e.g., muscle), ECM (e.g., collagen), cartilagenous material (e.g., joints such as knee, intervertebral discs, ears, noses, or the like), or the like, or combinations thereof; and the like; and combinations thereof. In one embodiment, the implant does not comprise a stent or a catheter. In another embodiment, the implant does not comprise a portion of soft tissue, ECM, or cartilagenous material, nor artificial soft tissue, ECM, or cartilagenous material. In a preferred embodiment, the useful implant is one commercially available from Synthes of Paoli, Pa., Stratec of Davos-Platz, Switzerland, and/or Norian of Cupertino, Calif.

Additionally or alternately, even implant tools that may not get implanted themselves but that are used in the procedure or surgery to introduce, to remove, to alter, and/or to repair an implant in vivo may be so coated, according to the present invention. Examples of such implant tools can include, but is not limited to, clamps, adductors, screwdrivers, drills, staplers, immobilizers, laparoscopic surgical instruments, scissors, scalpels, retractors, pick-ups, applicators (e.g., for bone cement and/or implants of similar consistency), guides, and the like, and combinations thereof.

Optionally but preferably (especially for implants that are non-polymeric and one or more of non-porous, non-absorbent in water or an organic liquid, and non-swellable in water or an organic liquid), the non-sterile implant is coated with a layer that is usually polymeric and/or that is one or more of porous, absorbent toward water or an organic liquid, and swellable in water or an organic liquid. The function of this coating, when present, is preferably to improve (in comparison to uncoated implants) the uptake of active agent (which is typically present in a solution of water or an organic liquid) to later form an active agent-loaded implant.

Exemplary coatings can include, but are not limited to, (co)polymers containing repeat units and/or (co)polymers made including precursors (i.e., monomers, dimers, oligomers, and the like, and combinations thereof) of aliphatic ethers (such as methylene oxide, ethylene oxide, propylene oxide, tetramethylene oxide, and the like, and copolymers and combinations thereof), aliphatic esters (such as caprolactones, e.g., ε-caprolactone; alkylene esters, e.g., ethylene adipate, butylene adipate, ethylene succinate, ethylene sebacate, ethylene glutarate, lactides/lactic acids (such as D-, L-, D,L-, and the like, and copolymers and combinations thereof), glycolides/glycolic acids, and the like, and combinations or copolymers thereof; and the like; and copolymers and combinations thereof), aromatic esters (such as ethylene terephthalate, butylene terephthalate, isophthalates, and the like, and copolymers and combinations thereof), aliphatic amides (such as lactams, e.g., propiolactam, caprolactam, laurolactam, and the like, and combinations and copolymers thereof; polyamides, nylon 6,6, nylon 6,9, nylon, 6,10, nylon 6,12, and the like; and copolymers and combinations thereof), siloxanes (such as alkyl and/or dialkyl siloxanes, methylsiloxane, dimethylsiloxane, methylethylsiloxane, and the like, and combinations and copolymers thereof), urethanes, and/or urethaneureas having hard segments made from at least diisocyanates (such as methylene diphenylene diisocyanate (MDI), methylene bis(cyclohexane isocyanate) (H₁₂MDI), isophorone diisocyanate (IPDI), phenylene diisocyanate, cyclohexane diisocyanate, toluene diisocyanate (TDI), methylcyclohexane diisocyanate, or the like, or combinations thereof) in combination with either diols (such as ethylene glycol, propylene glycol, butylene glycol, hexamethylene glycol, dihydroxybenzene, or the like, or a combination thereof) or diamines (such as ethylenediamine, propylenediamine, hexamethylenediamine, diaminocyclohexane, aniline, or the like, or a combination thereof) or both, optionally also including trifunctional and/or tetrafunctional components (such as triisocyanates, tetraisocyanates, triols, tetrols, triamines, tetramines, or the like, or a combination thereof) to chemically crosslink the (co)polymer system, alpha-olefins such as polyethylene (particularly UHMWPE), at least partially halogenated (particularly fluorinated) repeat units (e.g., vinyl halide, vinylidene halide, tetrahaloethylene, hexahalopropylene, perhaloalkoxy monomers such as those that form the commercial (co)polymer PFA available from DuPont of Wilmington, Del., perhaloester monomers, and the like, and combinations and copolymers thereof), momomers such as those that form the commercial (co)polymer SURLYN available from DuPont of Wilmington, Del., and the like), and the like, and combinations and copolymers thereof. Additionally or alternately, (co)polymers that are naturally occurring (or that are synthesized to approximate those that are naturally occurring) may be useful in the coating layer according to the invention, e.g., (co)polymers containing and/or made from hyaluronic acid and/or a salt thereof (such as lithium, sodium, potassium, magnesium, calcium, barium, or the like, or a combination thereof), collagen (such as type I, type II, or a combination thereof), or the like, or a combination thereof. In one particular embodiment, the coating layer contains or consists essentially of hyaluronic acid and/or a salt thereof (such as sodium hyaluronate). In another particular embodiment, the coating layer contains or consists essentially of a polylactic acid homopolymer or copolymer, e.g., poly(D,L-lactide), poly(D-lactic acid-co-L-lactic acid), or a combination thereof.

As is known in the art, the coating layer composition may optionally additionally include other conventional additives that may include, but are not limited to, leveling agents, various stabilizers, pH adjusting agents defoaming agents, cosolvents, and the like, and combinations thereof, particularly if compatible with the intended use of the coated implant.

The nature of the chemical and/or physical properties of the coating can be matched to the chemical and/or physical properties of the active agent. Optionally, where the coating surface has vastly different chemical and/or physical properties than the active agent and/or active agent solution, a treatment step may be performed after the implant is coated. When performed, the treatment step includes chemically altering (e.g., functionalizing, at least partially charging, ionizing, exciting, activating, or the like, or a combination thereof) the non-sterile implant surface to attain a more hydrophilic surface and/or a surface more prone to absorb, to adsorb, and/or to retain active agent, e.g., from solution (depending upon whether the active agent is in an aqueous solution or in a solution containing an organic solvent).

If necessary to assure proper adhesion and retention of the aforementioned coating layer to the non-sterile implant, an optional treatment step may be performed before the implant is coated. The treatment step may include chemically (e.g., functionalizing, at least partially charging, ionizing, exciting, activating, or the like, or a combination thereof) and/or physically (e.g., smoothing, roughening, ablating, or the like, or a combination thereof) altering the non-sterile implant surface. Additionally or alternately, the treatment step may include applying pressure to and/or adding water and/or an organic liquid to the coated layer, e.g., to ensure as complete a contact with the implant surface as possible.

If necessary or desired during and/or after the coating step, heat and/or reduced pressure may be applied to cure and/or dry the coating layer.

The non-sterile (coated) implant can then be sterilized by any convenient sterilization process known to those in the art. Exemplary sterilization processes include, but are not limited to, the application of heat (e.g., through increasing temperature and/or through contact with a heated object such as steam, i.e., autoclaving), irradiation (e.g., with UV light, gamma rays, or the like, or combination thereof), chemicals (e.g., exposure to ethylene oxide), or the like, or some combination thereof. Once the (coated) implant is sterilized, the sterilized implant may be packaged and/or shipped for later use.

After the coating process is completed, the coated implant can also be cleaned, in addition to being sterilized. Due to the absence of any active agents on/in the coated implant, a fairly extended shelf-life can be expected.

Alternately, a pre-packaged, sterile, coated implant (e.g., containing substantially no active agents or, if containing any active agent, the active agent being of a kind and/or in an available concentration insufficient for attaining the desired therapeutic goal) may be obtained, e.g., through commercial means and may alternately be used for convenience, instead of coating a commercially-obtained or pre-manufactured non-sterile implant.

The coating layer can be applied by any of a number of different processes known to those of skill in the art after fabrication of the non-sterile implant (e.g., by immersion or dip-coating, spray-coating, wipe-coating, injection molding, compression molding, plasma deposition, wet chemical reaction, or the like, or some combination thereof), or even during fabrication of the non-sterile implant (e.g., by co-molding, co-extrusion, simultaneous (co)polymerization, selective temperature profiling, or the like, or a combination thereof). If an appreciable amount of solvent(s) or other undesired volatile compounds is present during the coating process (e.g., in spray-coating, or particularly in dip-coating and wet chemical reaction, processes), an optional curing, or de-volatilization, step may be undertaken. Heating and/or applying a reduced pressure can help speed up de-volatilization of the solvent(s), and/or of any other volatile compounds present, in order to ensure proper setting, viscosity, hardness, and/or the like, in the coating layer. In addition, if a porous coating layer is desired, de-volatilization, e.g., by applying a combination of relatively high heat (typically not high enough to cause significant undesirable oxidation or degradation in either the coating layer or the underlying non-sterile implant, or both) with a relatively high vacuum (e.g., the combination of reduced pressure and the increased temperature can allow sufficient volatilization of the solvent(s) and/or of any other volatile compound(s) present so as to encourage those compounds to boil relatively rapidly and thus to cause pockets/bubbles of escaping gas to form; by adjusting the viscosity, hardness, and/or (co)polymer fraction in the coating layer, the bubbles can be induced to coalesce to provide a certain level of porosity in the coating layer). Although the combination of relatively high heat with a relatively high vacuum can generally result in a highly non-uniform coating layer and/or coating layer outer surface, such non-uniformity may be an acceptable effect of obtaining a porous coating.

It is noted that, in certain cases, it may not be desirable to coat the entire surface of the non-sterile implant (and/or implant tool). For example, for implants and tools that may typically encounter significant and/or repetitive shear and/or frictional stresses, but typically not those that encounter only extensional and/or compressive stresses, such that a coating layer would quickly delaminate or be worn away and thus would be of little practical value. Such circumstances may typically arise in, but are in no way limited to, the context of artificial joints or portions thereof; invertebral spinal discs; artificial muscles or portions thereof; springs; the legs of a staple or the end of a tack, nail, or pin that are driven into bone; the threading of a screw that is twisted into bone; and the like. In such circumstances, the coating layer may optionally be applied only to a selected portion of the implant (e.g., to the portion of the implant that preferably does not encounter significant and/or repetitive shear and/or frictional stresses).

The thickness of the coating layer is not necessarily constrained, nor is the thickness necessarily uniform on all portions of the implant (which are coated—the uncoated regions of the implant, if any, are specifically excluded from this uniformity consideration). Indeed, the coating layer (or the surface layer of the implant itself, if uncoated) is preferably thick enough to allow and/or facilitate physical and/or chemical association between the coating layer and the active agent for the active agent-containing solution). Thus, in one embodiment, the average thickness of the coating region is from about 10 nm to about 1 mm, depending upon the application for which the coated implant is to be used. In embodiments where a relatively thin coating layer is desired, the average thickness can be less than about 1000 nm, alternately from about 10 nm to about 1000 nm, from about 20 nm to about 500 nm, from about 10 nm to about 250 nm, from about 100 nm to about 1000 nm, or from about 250 nm to about 800 nm. In embodiments where a relatively thick coating layer is desired, the average thickness is from about 1 micron to about 1000 microns, alternately from about 1 micron to about 500 microns, from about 2 microns to about 300 microns, from about 5 microns to about 500 microns, from about 10 microns to about 800 microns, from about 50 microns to about 750 microns, or from about 250 microns to about 900 microns.

By loading the active agent into/onto/within the implant coating, the active agent can thereby be concentrated where it is most needed in vivo, while its presence, and consequently its effect, throughout the rest of the body can thus be minimized. An “active agent,” as used herein, should be understood to mean an agent that exhibits or can be caused to exhibit a therapeutically or diagnostically beneficial effect in the body.

The active agent (while the term “active agent” is referred to in its singular form, without any intent to limit, it should be understood that this term refers equally to mixtures and/or complexes of multiple active agents, which are also considered to be part of the present invention) may be chemically and/or physically associated with the coating layer (or with the surface of the sterilized implant, if no separately added coating layer is desired or necessary) using any number of means known to those of skill in the art. Indeed, the active agent may be contacted with the coating layer by means similar to those used to deposit the coating layer (e.g., immersion or dip-coating, spray-coating, wipe-coating, wet chemical reaction, or the like, or some combination thereof). While the pure active agent itself is typically a liquid or a solid between about room temperature (let's say about 15° C.) and about human body temperature (about 37° C.), an advantage may be obtained by placing or obtaining the active agent in an acceptable carrier/solution (hereinafter “solution,” for convenience only and without intent to limit). The solution need not completely dissolve the active agent (although that may be desired in some embodiments of the present invention), but generally should sufficiently attain a viscosity sufficient to allow the active agent to physically and/or chemically associate with the coating layer (e.g. in some embodiments, a more viscous solution may be desired so as to attain a thicker coating such as in an immersion or dip-coating process; in other embodiments, a less viscous solution may be desired so as to attain a thinner coating such as in an immersion or dip-coating process or so as to attain better flow such as through the tubes/pipes of a spray-coating apparatus). Thus, precipitated solutions, colloidal solutions, suspensions, emulsions, latices, flocculated solutions, agglomerated solutions, supersaturated solutions, and the like can be acceptable substitutes for substantially and/or completely dissolved active agent solutions.

There are many potential active agent categories for loading in/on the (coating layer of the) implants according to the present invention. Which category of active agents, and in fact which particular active agent, to employ will typically depend upon the goal to be attained by the active agent and optionally but preferably also upon the application for which the implant is to be used. For instance, where the implant is associated with a bone injury (e.g., bone screw, bone plate, a bone replacement composition, a protective membrane for bone replacement composition, or the like), a useful active agent may include an osseointegrative, osteoconductive, and/or osteoinductive agent (such as a morphogenic protein, a mitogenic protein, or a combination thereof), an antibacterial compound, an angiogenesis agent, an antiinflammatory agent, a nutrient, or the like, or a combination thereof, optionally depending upon a particular, patient or surgical need. A non-exclusive exclusive list of active agents follows: antibacterials, antivirals, antimicrobials, angiogenesis agents, antiinflammatories, anticancer agents, antiproliferative agents, anticlotting agents, antioxidants, antifungals, analgesics, antiseptics, bioabsorbability/bioresorbability enhancers, bisphosphonates, calcitonins, chemotherapeutics, clotting agents, drugs for treating pain, immune system boosters, immunosuppressants, immunomodulators, nutrients (e.g., vitamins), osteoclast inhibitors, conductors, osteoinductors, osseointegrative agents, statins, vasodilators, vasoconstrictors, and combinations thereof. Other additionally or alternately acceptable active agents and active agent categories can be found, e.g., in U.S. Pat. No. 6,221,383, the disclosure of which is hereby incorporated by reference.

In a preferred embodiment, the active agent includes an antibacterial agent, an antiviral agent, an osseointegrative, osteoconductive, and/or osteoinductive agent, or a combination thereof. In another preferred embodiment, particularly useful in subjects having osteoporosis, the active agent contains or consists essentially of mitogenic growth factors such as IGF and/or PDGF, morphogenic growth factors such as TGF and/or BMP, osteoclast inhibitors such calcitonin and/or bisphosphonates, antiinflammatories, and any combination thereof.

Alternately, the active agent can include any of a number of therapeutic or diagnostic agents that may be used for a variety of purposes, including improving the biocompatibility of the medical device. While traditional (or “sense”) oligonucleotides can generally function to increase and/or enhance desired protein expression, antisense oligonucleotides may be used to improve biocompatibility of the medical device, where the antisense oligonucleotide inhibits cell migration, inhibits synthesis of extracellular matrix proteins or growth factors, or induces apoptosis. Suitable antisense oligonucleotides include those described in U.S. Pat. Nos. 5,470,307, 5,593,974, and 5,756,476, and in Uhlmann et al., “Antisense Oligonucleotides: A New Therapeutic Principle” Chemical Reviews, 90(4), 544-579 (1990), the disclosures of each of which are hereby incorporated by reference in their entireties. The antisense oligonucleotides may be modified with avidin or biotin, or to contain hydrophobic groups such as cholesterol, to facilitate cellular uptake and prevent degradation by nucleases.

Similarly, extracellular matrix proteins may be used to improve biocompatibility of the medical device, or inhibit or prevent restenosis. Extracellular matrix proteins, such as fibronectin, laminin, collagen, and vitronectin, or synthetic peptide analogues of extracellular matrix proteins, have an amino acid sequence which contributes to cell adhesion. Synthetic peptide analogues of extracellular matrix proteins can also be used, which analogues retain their biological function but have a lower molecular weight and different solution properties. The extracellular matrix proteins or peptides are believed to attract migrating cells within the patient, and thus inhibit restenosis by preventing the cells from accumulating in the arterial lumen. Additionally, by attracting migrating cells, they are believed to facilitate integration with tissue of implanted devices and wound healing, and the uptake by cells of other therapeutic agents bound to the device surface. Additionally, the extracellular matrix proteins bound to the device surface may facilitate in vitro seeding of endothelial cells to the device prior to implantation or introduction of the device within the patient. In one embodiment, the extracellular matrix protein vitronectin can be bound to the device surface, and an antibody to the B1 integrin subunit can be bound to the device surface or can be delivered locally or systemically. This antibody has been shown to block cellular adhesion to all extracellular matrix proteins except vitronectin, thereby enhancing the adhesive power of the modified device surface.

Similarly, nitric oxide donor drugs may be used to improve biocompatibility of a medical device, and may also prevent or inhibit platelet aggregation and promote wound healing. Additionally, nitric oxide donor drugs may be used as a vasodilator relaxing smooth muscles, e.g., of a vessel prior to, during, and/or after angioplasty or stent placement. A variety of suitable nitric oxide donor drugs can be used including, but not limited to, nitric oxide-polyamine complexes, 2-methyl-2-nitrosopropane, S-nitroso-N-acetyl-D,L-penicillamine, 3-morpholoinosydoimine, sodium nitrate, s-nitrosoglutathione, sodium nitropnisside, and nitroglycerine. The structure and mechanisms of suitable nitric oxide donor drugs are disclosed, for example, in U.S. Pat. No. 5,650,447, the disclosure of which is also incorporated by reference in its entirety.

In the embodiment of the coating of the invention having a therapeutic or diagnostic agent bound to the medical device surface, directly or via a linking agent, the coating of the invention can provide localized delivery of the therapeutic or diagnostic agent. Similarly, the coating of the invention can also improve the residence time of the therapeutic or diagnostic agent. By binding the agent to the device, the rapid clearance from the bloodstream of the therapeutic or diagnostic agent, as for example when the body's immune system phagocytizes the therapeutic agent or a liposome containing the agent, can be avoided.

In one embodiment of the invention, release of the therapeutic or diagnostic agent within the patient from the medical device surface is provided by the coating of the invention. Such release of the therapeutic agent from the device surface may be desirable as occurring over a viable dosage period, e.g., a time release or extended release formulation. For example, an antisense oligonucleotide may be bound to the base coat by binding the antisense oligonucleotide to a sense oligonucleotide via Watson-Crick base-pairing. However, when the complementarity of the sense sequence is varied, the dissociation constant of the base-pair bond may be modulated, to thereby control the release of the antisense oligonucleotide from the device surface. Similarly, the avidin or biotin moiety of an avidin-biotin linking agent may be chemically altered to decrease the binding constant and thereby tailor the in vivo half life of the avidin-biotin complex.

In an alternate embodiment, the therapeutic or diagnostic agent includes, but is not limited to: proteins; peptides; oligonucleotides, antisense oligonucleotides; cellular adhesion promoting proteins or peptides including extracellular matrix proteins; polysaccharides such as heparin, hirudin, hylauronan, and chondroitin; nitric oxide donating compounds; vascular growth factors such as VEGF; antitumor agents such as Taxol, Paclitaxel, Carboplatin, and Cisplaten; and analogs, derivatives, and mixtures thereof. For example, paclitaxel (taxol) derivatives that may be suitable for use in the present invention can include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

In a preferred embodiment, the active agent comprises an osteogenic protein, preferably present in an amount sufficient to induce formation and/or regeneration of the desired replacement tissues (e.g., which can include not only bone but also marrow, blood vessels, extracellular matrix materials such as collagen, or the like, or, a combination thereof). The presence of the active agent can advantageously permit, facilitate, catalyze, and/or encourage regeneration of the tissues within the subject, including plural, tissues of appropriate size, interrelationship, and function. Exemplary osteogenic proteins believed to be useful as active agents according to the present invention described below and have been previously described in, e.g., U.S. Pat. Nos. 4,968,550, 5,258,494, and 5,266,683, the disclosures of each of which are incorporated by reference herein. The osteogenic protein be, for example, any of the known bone morphogenetic proteins and/or equivalents thereof described herein and/or in the art and can include naturally sourced material, recombinant material, and/or any material otherwise produced which is capable of inducing tissue morphogenesis.

In addition to osteogenic proteins, various growth factors, hormones, enzymes, therapeutic compositions, antibiotics, or other bioactive agents also can be adsorbed onto, absorbed into, and/or impregnated within, the coating layer such that they can advantageously be released over time when implanted. Thus, various known growth factors such as EGF, PDGF, IGF, FGF, TGF-α, TGF-β, the like, or any combination thereof, can be released in vivo.

Other often-complementary active agents include, but are not limited to, chemotherapeutic agents, insulin, enzymes, enzyme inhibitors, chemotactic-chemoattractant factors, and the like, and combinations thereof.

Osteogenic proteins useful in the compositions and methods according to the present invention include the family of proteins having endochondral bone activity when implanted in subject (e.g., such as a mammal) in association with an implant (coating layer) and that can comprise a subclass of the “super family” of “TGFP-like” proteins. Naturally-found osteogenic proteins in their mature, native forms are typically glycosylated dimers generally having an apparent molecular weight of about 30-36 kDa, e.g., as determined by SDS-PAGE. When reduced, the 30-36 kDa protein gives rise to two glycosylated peptide subunits having apparent molecular weights of about 16 kDa and 18 kDa. In their reduced states, the proteins typically exhibit no detectable osteogenic activity. The unglycosylated protein, which also has osteogenic activity, has an apparent molecular weight of about 27 kDa. When reduced, the 27 kDa protein gives rise to two unglycosylated polypeptides having molecular weights of about 14 kDa to 16 kDa, which are capable of inducing endochondral bone formation in a mammal. Additionally or alternately, variants of these proteins are also contemplated, such as those disclosed in U.S. Pat. No. 6,656,517, the disclosure of which is hereby incorporated by reference herein.

In one embodiment, the osteogenic protein can comprise OP1 or an OP1-related sequence. Useful OP1-related sequences include those recited in U.S. Pat. Nos. 5,011,691, 5,018,753, and 5,266,683, in Ozkaynak et al., (1990) EMBO J., 9:2085-2093, and in Sampath et al., (1993) PNAS, 90: 6004-6008. OP-1 related sequences can also include xenogenic homologs, e.g., 60A, from Drosophila (Wharton et al., (1991) PNAS, 88:9214-9218) and proteins sharing greater than 60% identity with OP1 in the C-terminal seven cysteine domain, preferably at least 65% identity. Examples of OP1-related sequences can include, but are not limited to, BMP5, BMP6, its species homolog Vgr-1 (Lyons et al., (1989) PNAS 86:4554-4558, Celeste et al., (1990) PNAS, 87:9843-9847, and International Publication No. WO93/00432), and OP-2 (Ozkaynak et al., (1992) J. Biol. Chem. 267:13198-13205), as well as combinations thereof. As will be appreciated by those having ordinary skill in the art, chimeric constructs readily can be created using standard molecular biology and mutagenesis techniques combining various portions of different morphogenic protein sequences to create a novel sequence, and these forms of the protein also are contemplated herein.

Alternatively, osteogenic polypeptide chains can be synthesized chemically using conventional peptide synthesis techniques well known to those having ordinary skill in the art. For example, the proteins may be synthesized intact or in parts on a solid phase peptide synthesizer, using standard operating procedures. Completed chains can then be deprotected and purified by HPLC (high pressure liquid chromatography). If the protein is synthesized in parts, the parts may be peptide bonded using standard methodologies to form the intact protein. In general, the manner in which the osteogenic proteins are made can be conventional and does not form a part of this invention.

Active agent carriers typically have some interaction with the active agent and can serve to dilute the concentration of the active agent, if desired, but generally do not affect the therapeutic and/or diagnostic effectiveness of the active agent in vivo. However, active agent carriers may optionally facilitate association of the active agent with the coated implant surface, e.g., through a physical and/or chemical association both with the active agent and with the coated implant surface. Carriers, as used herein, include, but are not limited to, adjuvants, excipients, solutions, emulsions, suspensions, colloidal phases, slurries, encapsulants, or the like, or a combination thereof. For example, where an extended dosage time and/or a time release formulation is desired, the active agent can be present in a first phase that is encapsulated by a second phase water-in-oil-in-water emulsions, oil-in-water-in-oil emulsions, microsphere encapsulation, micelle encapsulation, or the like, or a combination thereof.

Examples of active agent carriers can include, but are not limited to, water, saline, buffered aqueous solutions, supercritical carbon dioxide, polar organic solvents, non-polar organic solvents, and the like, and combinations thereof. In one preferred embodiment, the active agent can be present in a solution or slurry containing water.

Polar organic solvents include, but are not limited to: alcohols such as ethanol, propanol, isopropanol, and the like; alkylene glycols such as ethylene glycol, oligomeric poly(ethylene oxide) glycols, butanediol, and the like; multiply hydroxy-functional compounds such as glycerol and the like; aldehydes such as acetaldehyde, formaldehyde, and the like; ketones such as acetone, methyl ethyl ketone, methyl isobutyl ketone, and the like; amines such as mono-, di-, and/or tri-substituted alkylamines; amides such as dimethylformamide, dimethylacetamide, formamide, acetamide, acrylamide, and the like; carboxylic acid-functional compounds such as acetic acid, citric acid, and the like, as well as salts and/or esters thereof; halogenated hydrocarbons such as chloroform, methylene chloride, trichloroethane, bromoform, and the like, but preferably excluding chlorofluorocarbons and perhalohydrocarbons and optionally excluding halogenated aromatic compounds; sulfur-containing compounds such as dimethylsulfoxide and the like; fatty acids such as oleic acid, stearic acid, linoleic acid, behenic acid, palmitic acid, myristic acid, caproic acid, caprylic acid, capric acid, lauric acid, palmitoleic acid, and the like, as well as esters thereof such as diglycerides, triglycerides, and the like; natural or synthetic oils such as corn oil, canola oil, olive oil, sunflower oil, safflower oil, flaxseed oil, grapeseed oil, rapeseed oil, cottonseed oil, linseed oil, sesame oil, peanut oil, and the like; compounds containing more than one type of polar functional group enumerated herein such as citric acid; and the like; and combinations thereof.

Non-polar organic solvents include, but are not limited to: straight or branched alkanes such as pentane, hexanes, decanes, dodecanes, mineral spirits, oligomeric poly(alpha-olefin)s, and the like; cyclic alkanes such as cyclohexane and the like; straight or branched alkenes such as hexenes, butadiene, hexadiene, octadiene, septatriene, octatriene, and the like; cyclic alkenes such as cyclohexadiene, cyclooctatriene, norbornene, and the like; and the like; and combinations thereof.

When present with a carrier, the relative concentration of active agent in solution can advantageously be sufficient to permit and/or facilitate adsorption by, absorption into, uptake by, and/or bonding with the coating layer (and/or the exposed surface layer of the implant). Such active agent concentration can be expressed in weight percentage terms (e.g., from about 0.01% to about 75%, from about 0.1% to about 50%, from about 0.05% to about 20%, from about 0.2% to about 10%, from about 0.5% to about 25%, from about 0.05% to about 5%, or from about 1% to about 40%) or can alternately be expressed in terms of molarity (e.g., from about 0.0001M to about 5M, from about 0.001M to about 2M, from about 0.0005M to about 1M, from about 0.01M to about 0.7M, from about 0.05M to about 1.5M, from about 0.001M to about 0.5M, or from about 0.1M to about 3M), based on the solution/carrier composition, separate from the implant.

Any methods known to those of skill in the art for sterilizing solutions can be utilized in the method according to the present invention. One preferred example of sterilizing a solution includes filtering the solution through an appropriate filter/filtration apparatus (e.g., containing a filter having pore sizes not larger than about 0.45 microns, alternately not larger than about 0.22 microns, which are widely available commercially through a variety of sources). This liquid-based filtration method typically requires that the viscosity of the solution be manageable so that the pressure necessary (if any) to allow the solution to pass through the filter and/or requires that the active agent be sufficiently soluble in the solution so that any active agent-related solids therein have a diameter no larger than about the maximum pore size of the filter.

In addition, it is noted that the vessel in which the active agent-containing solution is held must also be sterilized to assure that the active agent-containing solution remains sterile throughout the method according to the invention. Similarly, in a process using an apparatus, each of the components of the apparatus must also be sterilized to assure that the active agent-containing solution remains sterile throughout the method according to the invention. These sterilization processes can be performed using any of the appropriate sterilization techniques described herein and/or known to those of skill in the art.

The active agent (and its optional carrier) can be handled separately in a vessel, for instance, according to general methods with which hospitals are acquainted. Just prior to implantation, the sterile coated implant can, in one embodiment, be immersed in the vessel in order to allow the coating layer of the implant to chemically and/or physically associate with the active agent and/or the active agent solution.

The active agent loading level (as well as the loading level of the solvent/carrier, if desired) in/on the implant can be easily and precisely adjusted by controlling the concentration of the active agent (and optionally the solvent/carrier concentration as well) in the solution, by carefully choosing the chemical nature of the solvent/carrier with an eye toward its compatibility or incompatibility with the coating layer material/surface, and/or by controlling the coating layer exposure time thereto.

The active agent loading level in/on the implant (coating layer) can advantageously be sufficient to permit, facilitate, catalyze, and/or encourage the expression of the desired active agent effect(s) and/or the attainment of the desired therapeutic/diagnostic result(s) in vivo when implanted in a subject. Similarly, if the solvent/carrier, independently or in conjunction with the active agent, permits, facilitates, catalyzes, and/or encourages the expression of the desired active agent effect(s) and/or the attainment of the desired therapeutic/diagnostic result(s) in vivo, then its concentration within the implant (coating layer) may be controlled as well.

Once sufficiently loaded with active agent, the implant can subsequently be maneuvered into position within a patient and (optionally permanently) implanted. Preferably, the method according to the present invention includes substantially contemporaneously loading the sterile, coated implant with an active agent and implanting the sterile, coated, active agent-loaded implant into a subject. “Substantially contemporaneously,” as used herein, should be understood to mean that the step of loading the sterile, coated implant with an active agent occurs at a time from immediately before to a reasonable time before implanting the sterile, coated, active agent-loaded implant into a subject. In one embodiment, the time between forming the active agent-loaded sterile implant and implanting the active agent-loaded sterile implant into a subject can be from about 20 seconds to about 16 hours, alternately from about 1 minute to about 12 hours, from about 20 seconds to about 1 hour, from about 30 seconds to about 8 hours, or from about 45 seconds to about 3 hours.

The term “at least partially chemically and/or physically associates with,” which is used herein to describe the interaction of the active agent (and optionally also the solvent/carrier) with the coating layer material and/or surface of the implant, can be defined broadly. Chemical associations, which include various levels of hydrogen-bonding, can range throughout the spectrum from relatively strong associations (e.g., chemical bonds and ionic charge-related attractions/repulsions) to relatively weak associations (e.g., intermolecular interaction based on partial electronic charge distributions, or mild polarity, and secondary intramolecular electronic structure interactions such as alignment of p-orbitals or empty d- or f-orbitals that can lead to intermolecular complexes). Physical associations can also range from relatively strong associations (e.g., co-crystallinity or co-crystallization, entanglements between relatively high molecular weight materials, and high levels of co-alignment or co-orientation) to relatively weak associations (e.g., low levels of co-alignment or co-orientation, van der Waals forces, and alteration of β-, γ-, and/or δ-phase transitions like a glass-amorphous liquid transition in polymers/oligomers such as through plasticization or the like). While certain types of relatively strong interactions between the implant (coating layer) and the active agent and/or active agent solution can be undesirable in circumstances where immediate or relatively quick active agent release in vivo is desired, such strong interactions may be desirable in other circumstances such as where relatively slow or extended/time release in vivo is desired. Thus, the desired release characteristics, as well as the uptake/loading characteristics, of the active agent (and/or solution/carrier) can be controlled by controlling the strength or weakness of the interactions within the system.

Another aspect of the invention relates to the sterile, coated, active agent-loaded implant formed according to the method of the invention described above.

Another aspect of the invention relates to a kit comprising (1) a sterile, coated implant formed according to the method of the invention described above and (2) a sterile solution/carrier comprising an active agent according to the invention.

While a particular form of the invention has been illustrated and described, it will also be apparent to those skilled in the art that various modifications can be made without departing from the spirit and scope of the invention.

### EXAMPLES

The following prophetic Example is intended to be merely indicative of a certain embodiment according to the invention and thus should not be construed to limit the scope of the invention or the claims in any way, nor be construed as a particularly preferred embodiment according to the invention.

A pre-fabricated intramedullary nail (e.g., a distal or trochaic femoral nail such as those commercially available under the tradenames DFN and TFN, respectively, from Synthes-Stratec of Paoli, Pa.) is used as the implant. The implant surface is then cleaned (and optionally treated to induce an at least partially functionalized implant surface, such as a hydroxyl-containing surface, e.g., by exposure to plasma in an oxidizing atmosphere, to facilitate stronger interaction with the coating layer) in preparation for application of the coating layer. The cleaned (and optionally functionalized) implant is then coated with a layer of either poly(D,L-lactide) (“pDLLA”) or hyaluronic acid that is optionally at least partially neutralized with sodium (collectively, “HA”), e.g., by a solution-coating method (e.g., using an aqueous solution for the HA coating layer or a polar organic solution for the pDLLA coating layer). The implant may then be treated to dry/cure the coating layer, e.g., though application of heat to form a stable coating layer on the implant surface. Afterwards, the coated implant is then subject to sterilization, e.g., through irradiation, and optionally packaged for later use.

Separately, an active agent solution is prepared having an osseointegrative, osteoconductive, and/or osteoinductive agent (e.g., growth factors such as IGF, PDGF, TGF; or a combination thereof) in a carrier, preferably including or consisting essentially of water. The active agent solution can then be sterilized, e.g., by filtration, and optionally packaged for later use.

The sterile coated implant can then be loaded with active agent by dipping/immersing the sterile coated implant in the sterile active agent solution for a time sufficient to achieve adequate absorption/adsorption of at least a portion of the active agent(s) (and optionally also at least a portion of the carrier) into/by the coating layer. For example, the dipping/immersion may take from about 10 seconds to about 30 minutes or from about 30 seconds to about one hour.

The active agent-loaded sterile implant can then be implanted into a patient relatively soon after active agent loading, e.g., within about 2 hours of loading. If there is a significant time delay (e.g., more than about 5-10 minutes) between the loading of the active agent-loaded and the implantation of the active agent-loaded sterile implant into the patient, the active agent-loaded sterile implant may be placed and/or isolated within a sterile area or enclosure, so that its sterility can be maintained. Implantation may be accomplished by standard techniques and using standard means.

Further, it should be understood that variations and modifications within the spirit and scope of the invention may occur to those skilled in the art to which the invention pertains. Accordingly, all expedient modifications readily attainable by one versed in the art from the disclosure set forth herein are within the scope and spirit of the present invention and are to be included as further embodiments. The scope of the present invention is accordingly defined as set forth in the appended claims.

What is claimed is:

1. A method of forming a sterile active agent-loaded implant comprising:
providing a sterile implant having a surface that is capable of associating with an active agent;
providing a sterile composition comprising an active agent, and
contacting the sterile implant surface with the sterile active agent-containing composition, so that the sterile implant at least partially physically associates, at least partially chemically associates, or both, with the active agent to form an active agent-loaded sterile implant.

2. The method of claim 1, wherein the sterile active agent-containing composition comprises a carrier for the active agent, a solvent for the active agent, or both.

3. The method of claim 2, wherein the solvent comprises water.

4. The method of claim 2, wherein the active agent comprises an antibacterial agent, an antiviral agent, an osseointegrative, osteoconductive, and/or osteoinductive agent, a mitogenic growth factor, a morphogenic growth factor, an osteoclast inhibitor, an antiinflammatory agent, or a combination thereof.

5. The method of claim 1, wherein the sterile implant comprises a coating layer, which layer comprises hyaluronic acid, a hyaluronate salt, a (co)polymer containing alkylene oxide repeat units, or a combination thereof.

6. The method of claim 4, wherein the sterile implant comprises a coating layer, which layer comprises hyaluronic acid, a hyaluronate salt, a (co)polymer containing alkylene oxide repeat units, or a combination thereof.

7. The method of claim 3, wherein the sterile implant comprises a coating layer, which layer comprises a hydrophilic (co)polymer, a water-swellable (co)polymer, or both.

8. The method of claim 1, wherein providing the sterile implant comprises sterilization through exposure to heat, radiation, chemical treatment, or a combination thereof.

9. The method of claim 1, wherein providing the sterile active agent-containing composition comprises sterilization through exposure to heat, filtration through a sub-micron filter, or a combination thereof.

10. The method of claim 3, wherein providing the sterile active agent-containing composition comprises sterilization through exposure to heat, filtration through a sub-micron filter, or a combination thereof.

11. A method of implanting sterile active agent-coated material, a sterile active agent-coated device, or both, comprising:
performing the method according to claim 1 to form active agent-loaded sterile implant; and
within about 3 hours after forming the active agent-loaded sterile implant, implanting the active agent-loaded sterile implant into a subject.

12. The method of claim 11, wherein the sterile active agent-containing composition comprises a carrier for the active agent, a solvent for the active agent, or both.

13. The method of claim 12, wherein the solvent comprises water.

14. The method of claim 12, wherein the active agent comprises an antibacterial agent, an antiviral agent, an osseointegrative, osteoconductive, and/or osteoinductive agent, a mitogenic growth factor, a morphogenic growth factor, an osteoclast inhibitor, an antiinflammatory agent, or a combination thereof.

15. The method of claim 11, wherein the sterile implant comprises a coating layer, which layer comprises hyaluronic acid, a hyaluronate salt, a (co)polymer containing alkylene oxide repeat units, or a combination thereof.

16. The method of claim 14, wherein the sterile implant comprises a coating layer, which layer comprises hyaluronic acid, a hyaluronate salt, a (co)polymer containing alkylene oxide repeat units, or a combination thereof.

17. The method of claim 13, wherein the sterile implant comprises a coating layer, which layer comprises a hydrophilic (co)polymer, a water-swellable (co)polymer, or both.

18. The method of claim 11, wherein providing the sterile implant comprises sterilization through exposure to heat, radiation, chemical treatment, or a combination thereof.

19. The method of claim 11, wherein providing the sterile active agent-containing composition comprises sterilization through exposure to heat, filtration through a sub-micron filter, or a combination thereof.

20. The method of claim 13, wherein providing the sterile active agent-containing composition comprises sterilization through exposure to heat, filtration through a sub-micron filter, or a combination thereof.
